(19) United States
(12) Patent Application Publication
(10) Pub. No.: US 2010/0047211 A1
(43) Pub. Date: Feb. 25, 2010

(54) METHOD OF GROWTH OF MESENCHYMAL CELLS UNDER NON-ADHERENT CONDITIONS FOR CLINICAL APPLICATIONS

(75) Inventor: Ian Mcniece, Lutherville, MD (US)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, Baltimore, MD (US)

(21) Appl. No.: 12/227,458

(22) PCT Filed: May 18, 2007
(86) PCT No.: PCT/US2007/011921

§ 371 Date: Oct. 23, 2009

Related U.S. Application Data
(60) Provisional application No. 60/801,661, filed on May 19, 2006.

(51) Int. Cl. A61K 35/12; C12N 5/00; A61P 21/00; A61P 25/00; A61P 9/00

(52) U.S. Cl. 424/93.7; 435/325; 435/382

(57) ABSTRACT

The invention provides methods for expanding mesenchymal stem cells (MSCs) in non-adherent cultures. The methods include the propagation of MSCs in or on non-adherent matrices. The invention further provides administration and the use of cells propagated by the method of the invention for administration and preparation of a therapeutic agent. The invention further provides kits including cells propagated by the methods of the inventions.

### RELATED APPLICATIONS

This application claims priority to Provisional Patent Application Ser. No. 60/801,661, filing date May 19, 2006, which is incorporated herein by reference in its entirety.

### GOVERNMENT SUPPORT

The invention was made under Grant Number CA088878 from the National Institutes of Health of the United States Government. The Government may have certain rights in relation to the application.

### FIELD OF THE INVENTION

This invention relates to methods of growth of mesenchymal cells under non-adherent conditions. The method allows for expansion of mesenchymal cells in suspension for research or therapeutic uses.

### BACKGROUND OF THE INVENTION

Mesenchymal stem cells are the formative pluripotential blast cells found inter alia in bone marrow, blood, dermis, and periosteum that are capable of differentiating into any of the specific types of mesenchymal or connective tissues (i.e. the tissues of the body that support the specialized elements; particularly adipose, osseous, cartilaginous, elastic, and fibrous connective tissues) depending upon various influences from bioactive factors, such as cytokines. In contrast to their hematopoietic counterparts, MSCs are adherent and can be expanded in culture. A number of U.S. Patents, e.g., U.S. Pat. Nos. 5,486,359; 5,591,625; 5,736,396; 5,811,094; 5,827,740; 5,837,539; 5,908,782; 5,908,784; 5,942,225; 5,965,436; 6,010,696; 6,022,540; 6,087,113; 5,858,390; 5,804,446; 5,846,796; 5,654,186; 6,054,121; 5,827,735; and 5,906,934 (all of which are incorporated herein by reference) disclose mesenchymal stem cells (MSC), which can be differentiated into several progenitor cells, for example muscle progenitor cells, connective tissue cell progenitor cells, or hepatic oval cells. Muscle progenitor cells differentiate further into cardiac, skeletal, and smooth muscle cells, whereas the connective tissue cell progenitor may differentiate into bone. The patents above further teach transgenic MSCs that carry a transgene, methods to promote differentiation of MSCs along specific paths, and therapeutic methods including the use of MSCs.

Human MSC (hMSC) can be identified by the presence or absence of specific cell surface markers (Pittenger and Martin, Circ. Res. 95:9-20, 2004, incorporated herein by reference). Typically, hMSC can be identified by the presence of surface markers CD13, CD29, CD44, CD49a, b, c, e, f, CD51, CD54, CD58, CD71, CD73, CD90, CD102, CD105, CD106, CDw11, CD120a, CD120b, CD123, CD124, CD126, CDC127, CD140a, CD166, P75, TGFb1R, TGFbIIR, HLA-A, B, C, SSEA-3, SSEA-4, D7; and the absence of surface markers CD3, CD4, CD6, CD9, CD10, CD11a, CD14, CD15, CD18, CD21, CD25, CD31, CD34, CD36, CD38, CD45, CD49d, CD50, CD62E, L, S, CD80, CD86, CD95, CD117, CD133, SSEA-1. Monoclonal antibodies specific to MSCs have also been identified (e.g., U.S. Pat. Nos. 5,486,359 and 5,811,094). However, most surface markers have been found inadequate as a means to identify stem cells because putative marker(s) may also be found on nonstem cells, or a particular marker may only be expressed on a stem cell at a certain stage or under certain conditions, such as CD34 on hematopoietic stem cells. Nevertheless, surface markers and other attributes are useful in characterizing a stem cell as isolated or cultured, to detect changes in cells in culture over time, and as a means to begin to understand its potential interactions with neighboring cells and the cell environment (Pittenger and Martin, Circ. Res. 95:9-20, 2004).

Mesenchymal stem cells can be isolated from a number of cells and tissues including bone marrow, embryonic yolk sac, placenta, umbilical cord, fetal and adolescent skin, and blood, and propagated in culture. Friedenstein et al. (Exp. Hematol. 4:267-274, 1976, incorporated herein by reference) initially isolated MSCs by their adherence to tissue culture surfaces. Similar methods for isolation of MSCs are still commonly used.

Plating studies indicate that MSCs are present at as a rare population of cells in bone marrow, representing about 0.001-0.01% of nucleated cells. However, MSCs can be readily expanded when grown at a very low plating density. Cotler et al. (Proc. Natl. Acad. Sci. USA. 97:3213-3218) noted that the number of colonies formed per 100 cells plated remained constant when the density of plating was varied from 0.5 to 12 cells per cm². However, the size of the colonies decreased markedly when the cells were plated at higher densities. Colonies of maximal size were obtained when cells were plated at 1.5 to 3.0 cells per cm². Plating at such low densities requires the use of large amount of tissue culture dishes, reagents, and space. Methods for culturing of MSCs in a less resource intensive manner is desirable.

Adult bone marrow-derived MSCs engraft in numerous organs and differentiate along tissue-specific lineages when transplanted into animals. They migrate into areas of muscle degeneration to undergo myogenic differentiation in immunodeficient mice. Injection of MSCs directly into infracted swine heart has been shown to induce myocardial regeneration and improved cardiac function (Shake et al., Ann. Thorac. Surg. 73:1919-1925, 2002). In addition, MSCs implantation has been demonstrated to induce therapeutic angiogenesis in a rat model of hindlimb ischemia through vascular endothelial growth factor (VEGF) production by MSCs (Al-Khaldi et al., Gene Ther. 10:621-629, 2003). In humans, bone marrow-derived MSCs have been used to regenerate the marrow microenvironment after myeloablative therapy. When introduced into the infracted heart, MSCs prevent deleterious modeling and improve recovery. Interestingly, implanted cells do not appear to expand after implantation when engrafted to tissue other than bone. Experiments using MSCs labeled with membrane dyes that would be diluted out after about 3 cell divisions were found months later even in repairing tissue (Pittenger and Martin, Circ. Res. 95:9-20, 2004).

Clinical trials have been initiated in several countries to test cell-based therapies for the treatment of the injured heart. However, no studies have demonstrated incorporation of MSCs into regenerating tissue. It has been suggested that the MSCs exert a therapeutic effect by paracrine actions exerted by the cells through the release of soluble factors (See e.g., Gnecchi et al., FASEB J. 20:661-669, 2006; and Nagaya et al., Circulation. 112:1128-1135, 2005). This theory is supported by data therein demonstrating that conditioned media from transgenic MSCs overexpressing the prosurvival gene Akt limits hypoxia-induced apoptosis and triggers vigorous spontaneous contraction of adult rat cardiomyocytes in culture. Moreover, injection of concentrated conditioned media from the Akt transgenic MSCs into infracted rat hearts significantly limited infarct size and improved ventricular function relative to controls (Gnecchi et al., 2006).

Studies have demonstrated that upon transplantation of cells into cardiac tissue (e.g., by injection) less than 3% of injected MSCs persist after 2 weeks (Mazhari & Hare, Nature Clinical Practice Cardiovascular Medicine 4: suppl 1; S21-S26, 2007). This may be due to the adherent culture methods used to culture the MSCs. MSCs in bone marrow are able to adhere to bone to allow for proliferation. No comparable surface is present in muscle or many other tissues in which MSCs have been demonstrated to be beneficial. Current culture methods select for cells that are able to adhere to culture dishes through repeated rounds of trypsinization. When transplanted into cardiac tissue for example, MSCs may fail to proliferate due to their inability to adhere to a cardiac tissue surface, minimizing the contribution of MSCs to regenerating tissue.

Methods of culture of MSCs that do not include adherence to a surface and/or reduce the need for multiple rounds of trypsinization for propagation of cells may improve the effects of MSC at sites of injury, for example, by providing cells that are more able to proliferate at the site of injury.

### SUMMARY OF THE INVENTION

The invention provides methods for the propagation of mesenchymal stem cells (MSCs) in non-adherent culture, eliminating the need for trypsinization in propagation of MSCs.

Accordingly, an aspect of the invention features a method for culturing MSCs under non-adherent conditions in or on a non-adherent matrix to obtain an expanded population of MSCs. The methods include formation of MSC spheres (MSCS) in or on several different non-adherent matrices, including incorporation of cells into biocompatible matrices such as Hydrogel and Matrigel™; culture of cells on or between layers of agarose; and culture of cells in Teflon® bags. After isolation of MSCs from a sample, the cells are propagated without treatment with trypsin after initial cell selection. MSCS are optionally mechanically manipulated, collected by centrifugation, and resuspended in fresh media for continued propagation, or resuspended in an appropriate buffer for administration to a subject.

An aspect of the invention features a method for therapeutic administration to a subject in need of treatment with MSCs comprising; i)obtaining MSCs, for example by isolating the cells from a sample, ii) culturing the cells in a non-adherent manner to generate an expanded population of cells, and iii) administering the cells to the subject. In an embodiment, the MSCs are administered to an individual having a condition or disease susceptible to treatment with MSCs

An aspect of the invention provides for the use of MSCs cultured under non-adherent conditions for use as a medicament for the treatment of a condition or disease susceptible to treatment with MSCs.

An aspect of the invention includes kits containing MSCs expanded under non-adherent conditions in appropriate packing material. In an embodiment, the kits further include reagents or materials for propagation of the cells under adherent and/or non-adherent conditions.

In some embodiments of the invention, the methods further include obtaining a sample that contains MSCs, and may further include isolating the MSCs to obtain a substantially purified sample of MSCs.

In some embodiments of the invention, culturing the MSCs increases the expansion of the cells by at least 2 fold, preferably at least 10 fold or 100 fold, more preferably 1000 fold, 10,000 fold, or 100,000 fold. In another embodiment of the first or second aspects of the invention, the MSCs are maintained in non-adherent culture for at least one week, preferably at least two weeks, at least a month, or at least two months.

In some embodiments of the invention, the cultured MSCs are suitable for administration to a subject, preferably a human subject.

In some embodiments of the invention, the MSCs are allogenic or autologous to the subject to whom the cells are administered.

In an embodiment, the MSCs may express classic surface markers including CD105, CD73 and CD90 but lack expression of CD34 or CD45.

### Definitions

By “administering”, “therapeutic administration” and the like is meant providing to a human patient a pharmaceutical preparation containing the MSCs, optionally in the form of MSC spheres or foci, or their progeny or derivatives in a suitable formulation. The preferred method of administration can vary depending on various factors, e.g., the components of the pharmaceutical preparation, site of the potential or actual disease, and severity of disease.

By “allogenic” is meant involving, derived from, or being individuals of the same species that are sufficiently unlike genetically to interact antigenically.

By “animal” is meant to be preferably a mammal. A mama can be human or non-human including, but not limited to laboratory and/or commercially important mammals, such as mouse, rat, rabbit, monkey, dog, cat, pig, cow, sheep, and goat.

By “autologous” is meant derived from the same individual or involving one individual as both donor and recipient.

By “cell culture” is meant grown outside of the body in a dish, flask, or other container in the presence of growth media Cell culture can be performed with transformed or immortalized cell lines. Cell culture can also be performed with “primary cells” removed from an animal, such as a mammal, and are not transformed or immortalized. Primary cells can be dividing or non-dividing cells. For example, the cells can be bone marrow cells, umbilical cord blood cells, or mesenchymal stem cells.

By a “condition or disease susceptible to treatment with MSCs” is meant a malady that has been demonstrated to be treated using MSCs, for example muscle disease, neural disease, and vascular disease. Theses diseases have been demonstrated to be susceptible to treatment with MSCs. For example, demonstrated therapeutic effects include those shown in U.S. Pat. No. 5,811,094 to promote connective tissue regeneration; U.S. Pat. No. 5,858,930 for repair of skin and soft tissue defects; U.S. Pat. No. 6,387,369 for cardiac muscle regeneration; U.S. Pat. No. 6,875,430 for treatment of immune responses in transplantation; U.S. Pat. No. 7,029,666 for muscle and connective tissue repair, U.S. Pat. No. 7,097,832 for enhancing blood vessel formation; and U.S. Pat. No. 7,160,724 for repair of the brain and spinal cord.

By “effective amount” is an amount sufficient to effect beneficial or desired clinical or biochemical results. An effective amount can be administered one or more times. For purposes of this invention, an effective amount is the amount of MSCs to effect beneficial engraftment of the cells.

By “engraftment” is meant the implantation of cells in the body, and/or replacement of lost or damaged cells with injected cells. The engrafted cells persist in a particular location over time following transplantation of the cells into a mammal (e.g., a human).

By the term “expanded population” is meant a population of cells, e.g., MSCs isolated from bone marrow or other tissue, wherein at least 50% of the cells have divided at least once.

A molecule is a “marker” of a desired cell type if it is found on a sufficiently high percentage of cells of the desired cell type, and found on a sufficiently low percentage of cells of an undesired cell type, such that one can achieve a desired level of purification of the desired cell type from a population of cells comprising both desired and undesired cell types by selecting for cells in the population of cells that have the marker. A marker can be displayed on, for example, 30%, 35%, 40%, 45%, 50%, 55%, 60%, 65%, 70%, 75%, 80%, 85%, 90%, 95%, 99% or more of the desired cell type, and can be displayed on fewer than 50%, 45%, 40%, 35%, 30%, 25%, 20%, 15%, 10%, 5%, 1% or fewer of an undesired cell type. It is preferred that a marker be displayed on 90% or more of a desired cell type, or on fewer than 10% of a desired cell type.

A desired cell type is negative for a cell surface-expressed marker or lacks expression of the marker if fewer than 50 marker molecules per cell are present on the cell surface of the desired cell type. Techniques for detecting cell surface-expressed marker molecules are well known in the art and include, e.g., flow cytometry. One skilled in the art can also use enzymatic amplification staining techniques in conjunction with flow cytometry to distinguish between cells expressing a low number of a marker molecule and cells that do not express the marker molecule (see, e.g., Kaplan, Front. Biosci. 7:c33-c43, 2002; Kaplan et al., Amer. J. Clin. Pathol. 116:429-436, 2001; and Zola et al., J. Immunol. Methods 135:247-255, 1990).

By “non-adherent matrix” is meant a material which cells can grow in, or on a material that prevents adhesion to a cell culture container surface. For example, growing cells in a non-adherent matrix (e.g., Hydrogel, BD Biosciences or Matrigel®, BD Biosciences) can prevent attachment to a cell culture container surface. MSCs may adopt their typical fibroblast-like shape on the matrices, but do not attach to the plastic culture surface. Alternatively a non-adherent matrix can be understood to be a matrix that the cells can grow on, but do not attach tightly to (e.g., agarose, or Teflon®). With such matrices, the MSCs retain a rounded, rather than fibroblast shape which they obtain when grown on plastic. In a preferred embodiment, the non-adherent matrix is preferably biocompatible such that it can be administered to a subject for transplant without -separation from the matrix. Alternatively, the matrix can be of a size, shape, and resiliency that readily allows for removal of the cells from the matrix (e.g., Teflon®) to allow the cells to be administered to a subject.

By “mesenchymal stem cell” (MSC) is meant an adherent stroma cell, for example from a biological sample such as bone marrow or umbilical cord blood, isolated by methods such as those provided herein and by U.S. Pat. Nos. 5,486,359; 5,654,186; 5,827,735; 5,858,390; 5,906,934; 5,908,784; 5,965,436; and 7,060,494. Such cells have been characterized by being multipotent stem cells that have the capacity to differentiate into osteoblasts, adipocytes and chondrocytes in vitro and express the surface antigens CD105, CD73 and CD90, but not CD45 or CD34 (Dominici et al, Cytotherapy 8:315-317, 2007)

By a “muscle cell” is meant a skeletal, smooth, or cardiac cell.

By “muscle disease” is meant a disease or disorder that affects or involves the musculature, e.g., cardiac, smooth, or skeletal muscles. Examples of muscle diseases include neuromuscular disease, e.g., muscular dystrophy (e.g., Duchenne muscular dystrophy (DMD), Becker muscular dystrophy (BMD), Limb-girdle muscular dystrophy, and congenital muscular dystrophy), congenital myopathy, and myasthenia gravis, cardiomyopathy, e.g., heart disease, aortic aneurysm (Marfan's disease), cardiac ischemia, congestive heart failure, heart valve disease, and arrhythmia, and metabolic muscle diseases.

By a “neural cell” is meant a neuron (e.g., a sensory neuron, a motor neuron, or an interneuron) or a support cell of the central or peripheral nervous system. Examples of neurons include pyramidal cells, Betz cells, stellate cells, horizontal cells, granule cells, Purkinje cells, spinal motor neurons, and ganglion cells. Examples of support cells include glial cells, oligodendroglial cells, astrocytes, satellite cells, microglial cells, and Schwann cells.

By “neural disease” is meant a disease or disorder that affects or involves the central or peripheral nervous system. Examples of neural diseases include multi-infarct dementia (MID), vascular dementia, cerebrovascular injury, Alzheimer's disease (AD), neurofibromatosis, Huntington's disease, amyotrophic lateral sclerosis, multiple sclerosis, stroke, Parkinson's disease (PD), pathologies of the developing nervous system, pathologies of the aging nervous system, and trauma, e.g., head trauma Other examples of neural diseases are those that affect tissues of the eye, e.g., the optic stalk, retinal layer, and lens of the eye, and the inner ear. In certain embodiments, the patient may have suffered a neurodegenerative disease, a traumatic injury, a neurotoxic injury, ischemia, a developmental disorder, a disorder affecting vision, an injury or disease of the spinal cord, or a demyelinating disease.

By “non-adherent culture” is meant herein as a method of propagation of cells in vitro as in a container in the presence of growth media in a manner in which the cells do not attach to the surface of the container such that a substantial portion of the cells can be removed from the surface of the container by mechanical manipulations that do not cause significant damage to the cells. It is understood that the cells can still be retained in or on a non-adherent matrix (e.g., on Hydrogel spheres) and be removed from the surface of the container. Such manipulations include, for example, gentle agitation, massage, or manual manipulation of the container, or rinsing the container with growth media As used herein, a substantial portion of the cells to be removed is at least 70%, preferably at least 75%, 80% or 85%, more preferably at least 90% or 95%. Manipulations that cause damage to the cells can be identified by determining the viability of the cells before and after manipulation, for example by trypan blue staining. Mechanical manipulations should cause damage to less than 20%, preferably less than 15%, or 10%, more preferably less than 5%, 2%, or 1% of the cells.

By “obtaining” as in “obtaining an agent” or “obtaining a cell” refers to purchasing, synthesizing, or otherwise procuring an agent or cell. Cells can be obtained, for example, from an animal including human and non-human animals. Cells can also be obtained from cell and tissue repositories.

By “prevent,” “preventing,” “prevention,” “prophylactic treatment” and the like is meant reducing the probability of developing a disorder or condition in a subject, who does not have, but is at risk of or susceptible to developing a disorder or condition. Prevention or prophylactic treatment can require administration of more than one dose of the compositions of the invention.

By “propagate”, “passage”, and the like is meant increasing the volume of a cell culture and/or decreasing the amount of cells in a specific culture volume by diluting cells in at least some fresh growth media to allow for maintenance and/or expansion of the cell population.

By “sample” or “biological sample” is meant any biological sample obtained from an individual, body fluid, cell line, tissue culture, or other source.

By “stem cell” or “pluripotent stem cell,” which can be used interchangeably, is meant a cell having the ability to give rise to two or more cell types of an organism.

By “subject” is meant a vertebrate, preferably a mammal, more preferably a human.

By “substantially purified” is meant that the desired cells (e.g., MSCs) are enriched by at least 30%, more preferably by at least 50%, even more preferably by at least 75%, and most preferably by at least 90% or even 95%.

By “transgene” is meant any piece of a nucleic acid molecule (for example, DNA) that is inserted by artifice into a cell transiently or permanently, and becomes part of the organism if integrated into the genome or maintained extrachromosomally. Such a transgene may include a gene that is partly or entirely heterologous (foreign) to the transgenic organism, or may represent a gene homologous to an endogenous gene of the organism. The transgene may be introduced into the organism from which the MSCs are isolated. Alternatively, the transgene may be introduced using viral vectors, such as retroviral vectors (See, e.g., Gnecchi et al., 2006).

By “transgenic cell” is meant a cell containing a transgene. For example, a cell transformed with an expression vector operably linked to a heterologous nucleic acid molecule can be used to produce a population of cells having altered phenotypic characteristics. A cell derived from a transgenic organism is also a transgenic cell so long as the cell contain the transgene.

By “transplant” or “transplanting” is meant administering one or more cells (or parts thereof), cell products, tissue, or cell culture products derived from cells that are grafted into a human host. For example, a transplant can include an MSC transplant.

By “treatment” is meant an approach for obtaining beneficial or desired clinical results. For the purposes of this invention, beneficial or desired clinical results include, but are not limited to, alleviation of symptoms, diminishment of extent of disease, stabilization (i.e., not worsening) of a state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” refers to both therapeutic treatment and prophylactic or preventative measures. Those in need of treatment include those already with the disorder as well as those in which the disorder is to be prevented. “Palliating” a disease means that the extent and/or undesirable clinical manifestations of a disease state are lessened and/or the time course of the progression is slowed or lengthened, as compared to a situation without treatment. Typically, the “treatment” entails administering an effective dose of MSCs to the patient to regenerate tissue.

By a “vascular cell” is meant an endothelial cell. Endothelial cells line the blood and lymph vessels and are present in and play a key role in the development of organs, such as the brain, heart, liver, pancreas, lungs, spleen, stomach, intestines, and kidneys.

By “vascular disease” is meant a disease or disorder that affects or involves the vasculature. Examples of vascular disease include peripheral vascular disease, peripheral arterial disease, venous disease (e.g., deep vein thrombosis), ischemia, cardiovascular disease, tissue organ engraftment rejection, or sequelae of ischemic reperfusion injury. In still another embodiment, the peripheral vascular disease is atherosclerosis, thromboembolic disease, or Buerger's disease (thromboangiitis obliterans). In a further embodiment, the cardiovascular disease is myocardial infarction, heart disease, or coronary artery disease.

As used herein, “a”, “an”, and “the” are understood to be either singular or plural unless otherwise obvious from context.

As used herein, “or” is meant to be inclusive unless otherwise obvious from context.

As used herein, ranges are understood to include all values within the range. For example, 1 to 50 is understood to mean 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50. A series of values are understood to represent a range, and thereby all of the values within the range unless otherwise obvious from context.

### BRIEF DESCRIPTION OF THE DRAWINGS

FIGS. 1A-1B are images of MSC harvested from plastic adherent culture of MSC by trypsinization and then cultured for 1 week in (A) plastic tissue culture dish, (magnification 100×), or (B) grown in a double layer agarose culture (magnification 100×), or cultured for 2 weeks (C) in liquid culture above a single layer of agarose to prevent adherence to plastic (magnification 100×)

FIG. 2A-2B is an image of MSC spheres generated in culture of MSCs in Teflon® bags (A) grown in culture for 2 weeks (mag 100×) and (B) for 6 weeks (mag 100×).

FIG. 3 is an image of proliferating MSCs in hydrogel for 2 weeks (mag 100×)

FIG. 4 is an image of MSCs in a tissue culture flask after seven passages in Teflon® bags and then transferred to a plastic culture flask. The MSC spheres adhered to the surface of the flask within 2 to 3 days and obtained a morphology essentially identical to that observed in cells passaged in adherent cultures (mag 100×).

### DETAILED DESCRIPTION

Mesenchymal stem cells have been demonstrated to be useful in the therapeutic methods for the repair and regeneration of tissue, especially muscle tissue, including cardiac tissue. This is somewhat surprising as MSCs have been demonstrated to be quiescent after injection, have low engraftment into tissue other than bone, and to have a very low persistence after injection.

Mesenchymal stem cells are adherent cells, and can be selected for growth in culture by their ability to adhere to tissue culture containers (i.e., plastic). In culture, cells are propagated by repeated rounds of trypsinization and replating, effectively selecting for cells that are adherent. The observed low level of engraftment and cell division in vivo may be due to the in vitro methods of propagation of the MSCs in adherent cultures, as no comparable surfaces are available in vivo, for example in muscle, vascular, and neural cells.

The invention provides methods for mesenchymal stem cells (MSCs) growth in non-adherent culture, eliminating the need for trypsinization in propagation of MSCs. The non-adherent culture methods of the invention allow for the propagation of MSCs that may more readily engraft into recipient tissue and be more viable for longer periods after transplant as they do not require a surface to which they can adhere to divide.

The non-adherent culture methods of the invention also allow for propagation of cells in a less resource intensive manner by allowing the cells to be grown in larger numbers in the same culture container area as the cells do not need to all grow in the same plane of the culture container as with adherent cells.

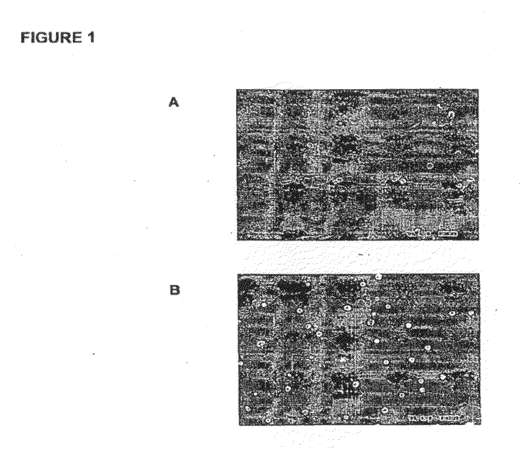

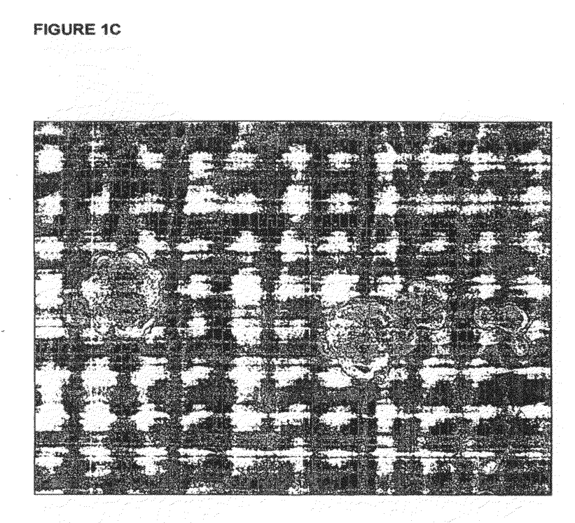

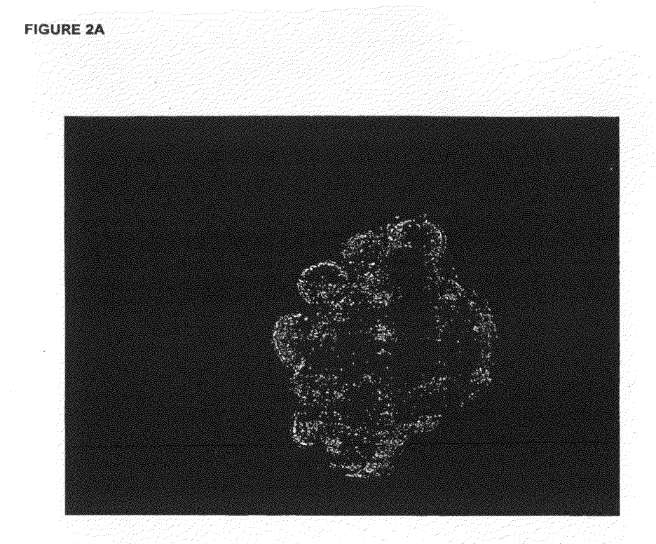

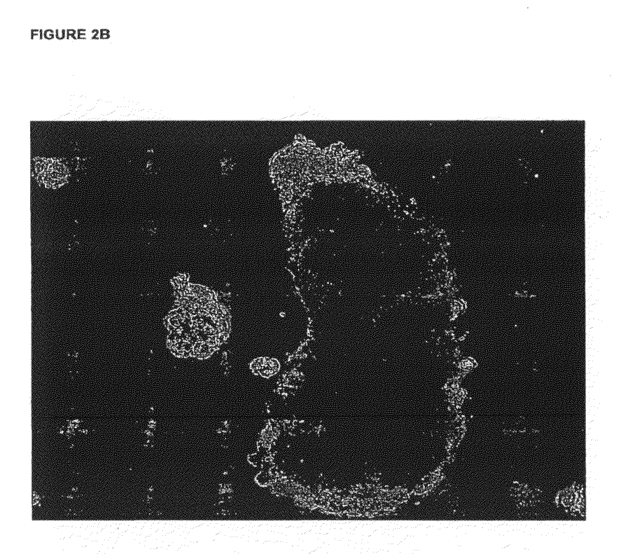

The invention provides culture methods that enable the generation of MSC in non-adherent foci in various support matricies. MSCs grown under these conditions can be passaged without trypsinization. Methods include growth of cells encapsulated in matrices such as Hydrogel and Matrigel®, on or between layers of agarose, or in Teflon® bags. Cells can grow in contact with the non-adherent matrices, but do not adhere to plastic culture containers. The lack of adherence to a surface is notable in the MSCs grown on agarose or in Teflon® bags as can be determined by the maintenance of their rounded shape. MSCs grown in adherent cultures on plastic adopt an elongated, fibroblastic shape (see, e.g., compare FIG. 1A with FIGS. 1B-1C and 2A-B).

Mesenchymal stem cells have been cultured for up to 10 passages and can be subcultured without the need of treatment with trypsin. The non-adherent cells express similar surface markers as cells grown under adherent conditions (e.g., CD105), and they maintain their ability to differentiate into multiple cell types. Optimal growth of the cells is stimulated by basic fibroblast growth factor (bFGF) and other growth factors including stem cell factor (SCF) and vascular endothelial growth factor (VEGF).

Growth of non-adherent MSCs in Teflon® bags provides an additional advantage for translation into therapeutic applications as the MSCs can be cultured by massaging the bag to detach the cells from the surface. When the MSCs are detached the can be maintained as MSC spheres by regular massaging of the bag and inversion of the bag for continued incubation. Performance of this manipulation about twice daily allows for the MSC spheres to increase in size, and for the MSCs to continue to proliferate and expand. The cells can readily be removed from the culture media by centrifugation and resuspension into an appropriate buffer for injection (e.g., phosphate buffered saline (PBS), physiological saline solution) without the need to remove the cells from a less sturdy non-adherent surface (e.g., Matrigel® or agarose) and without the use of trypsin which would need to be removed from the cells prior to administration.

It is understood that the initial source of and method of isolation of the MSCs to be grown by the culture methods of the invention is not a limitation of the invention. A number of methods of isolation of MSCs are known to those skilled in the art including, but not limited to, those set forth in U.S. Pat. Nos. 5,486,359; 5,654,186; 5,827,735; 5,858,390; 5,906,934; 5.908,784; 5,965,436; and 7,060,494.

It is further understood that the methods provided herein can be used to culture both wild-type and transgenic MSCs such as those taught, for example in U.S. Pat. No. 5,591,625 or in Gnecchi et al. (both incorporated herein by reference). Transgenic MSCs can be isolated from transgenic animals or can be transduced using vectors, including viral vectors, for the insertion of expression constructs into the cells.

Mesenchymal stem cells cultured by the methods of the invention can be used for any of a number of research or therapeutic purposes. For example, a number of therapeutic methods using MSCs are known, such as those taught in U.S. Pat. No. 5,811,094 for connective tissue regeneration; U.S. Pat. No. 5,858,930 for repair of skin and soft tissue defects; U.S. Pat. No. 6,387,369 for cardiac muscle regeneration; U.S. Pat. No. 6,875,430 for treatment of immune responses in transplantation; U.S. Pat. No. 7,029,666 for muscle and connective tissue repair; U.S. Pat. No. 7,097,832 for enhancing blood vessel formation; and U.S. Pat. No. 7,160,724 for repair of the brain and spinal cord (all of which are incorporated herein by reference).

Mesenchymal stem cells cultured by the methods of the invention can be used for the generation of cultured media to promote the growth of cells, for therapeutic uses, or for research purposes to identify secreted growth factors that may be responsible for the beneficial therapeutic effects provided by MSCs.

Mesenchymal stem cells cultured by methods of the invention can be incorporated into a kit including the cells in a container with appropriate packing material. The kit can further contain reagents and/or materials for culturing MSCs in adherent and/or non-adherent manner(s).

### Example 1

### Isolation of MSCs from Human Bone Marrow

Human bone marrow cells were obtained from normal donors following informed consent under an Institutional Review Board approved protocol. The mononuclear cell fraction of the bone marrow was isolated on a Ficoll gradient and plated in a T150 Corning (Acton, Mass.) tissue culture flask at 1-5×10⁶ cells/ml in α-MEM media containing 20% fetal calf serum (FCS). The cells were incubated in a humidified environment at 5% CO₂ at 37° C. The media was changed weekly. Adherent cells were grown in culture and passaged using trypsin when confluent.

### Example 2

### Culture of MSCs in Hydrogel

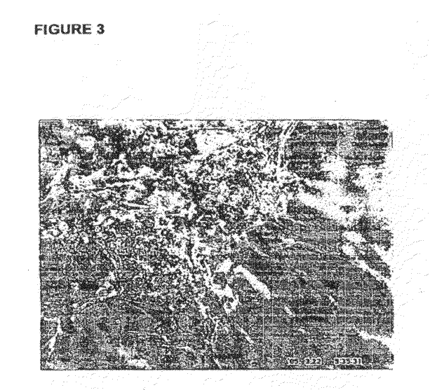

MSCs were isolated and propagated as set forth above. MSCs were collected from adherent, confluent cultures using trypsin and encapsulated in Hydrogel (Becton Dickson) following the manufacturer's instructions. The encapsulated MSCs were cultured in α-MEM+20% FCS in T75 culture flasks. At regular intervals, the non-adherent cells were passaged by removing the supernatant, centrifuging the Hydrogel/MCS mixture, and resuspending the cells in growth media. As shown in FIG. 3, MSCs encapsulated in the Hydrogel proliferated and maintained a fibroblast-like morphology. Cells encapsulated in Matrigel™ gave comparable results.

### Example 3

### Culture of MSCs in Agarose

Single layer agarose cultures were established in 100 mm culture dishes on preformed layers of 0.5% agarose for double layer, and 1% agarose for single layer agarose in α-MEM+30% FCS. MSCs were harvested from confluent cell cultures by trypsinization and resuspended in α-MEM+20% FCS. The MSCs were added in 10 ml of α-MEM+20% FCS above the agarose layer. The non-adherent cells were passaged by removing the supernatant from the agarose underlay. The cells were centrifuged and the supernatant discarded. The cells were resuspended in fresh media and replated over the agarose underlay. Double layer agarose cultures were generated by incorporating the cells into a top agarose layer (0.66%).

FIG. 1B shows cells cultured in a double layer agarose culture in the top agarose layer. The MSCs could be visualized as single, round cells. No proliferation was observed. However, when the cells were plated in a liquid phase in α-MEM+20% FCS on a lower layer of 1% agarose to prevent adherence, the MSC formed spheres and proliferated as shown in FIG. 1C. Cells were passaged multiple times.

### Example 4

### Culture of MSCs in Teflon®Bags

MSCs were harvested from confluent cell cultures by trypsinization and resuspended in 50 ml of α-MEM+20% FCS. The cells were placed in 100 ml Teflon® bags (American Fluoroceal Corp, Gaithersburg, Md.) and cultured. At weekly intervals the bags were harvested, the cells were centrifuged, resuspended in fresh media and placed into new Teflon® bags.

MSCs can be cultured by massaging the bag to detach the cells from the surface. When the MSCs are detached the can be maintained as MSC spheres by regular massaging of the bag and inversion of the bag for continued incubation. Performance of this manipulation twice daily allows for the MSC spheres to increase in size, and for the MSCs to continue to proliferate and expand (see, FIG. 2).

The culture methods have been replicated beginning with bone marrow harvested from pig. Non-adherent cultures of pig MSC have now been generated for animal studies. One hundred million non-adherent pig MSCs were generated after 3 weeks of culture in Teflon® bags.

### Example 5

### Adherence of Cells to Plastic After Culture Under Non-Adherent Conditions

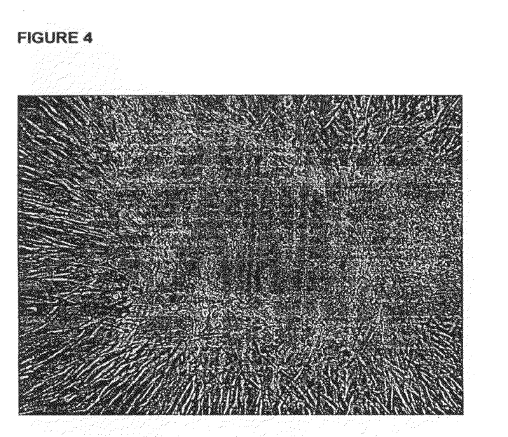

Culturing of cells under non-adherent conditions does not alter the ability of the MSCs to adhere when provided with an appropriate substrate. FIG. 4 shows cells grown in a tissue culture flasks after seven passages in Teflon® bags. The morphology of the cells appears to be identical to that of MSCs propagated continuously in adherent culture.

### Example 6

### Stimulation of MSCs by Growth Factors

The effect of several growth factors on MSC sphere proliferation were evaluated, including macrophage colony stimulating factor (M-CSF), vascular endothelial growth factor (VEGF), basic fibroblast growth factor (bFGF), stem cell factor (SCF), and media conditioned by 5637 cells (a human bladder carcinoma cell line that constitutively secretes functional cytokines). Optimal growth factors for of MSC spheres was addition of 10% 5637 conditioned media to the cells. However, as this source of growth factors is limited for clinical applications, the effects of recombinant growth factors were also analyzed. A combination of recombinant human bFGF (50 ng/ml) and recombinant human SCF (100 ng/ml) resulted in maximal proliferation of MSCs and sphere formation.

### Example 7

### Transplantation of Non-Adherent MSCs for the Treatment of Cardiac Infarction

MSCs are isolated from rat bone marrow by standard Ficoll gradient followed by adherent culture methods. After expansion of the cells, the culture is split. A portion of the cells are maintained in adherent culture, and a portion of the cells are transferred to Teflon® bags for propagation. Cells in Teflon® bags are manipulated twice daily to promote growth of MSC spheres, and media is changed as needed. Adherent cells are propagated using trypsin as needed. Cells can include a marker such as GFP or beta-galactosidase to facilitate identification of the transplanted cells at the end of the experiment. Cells are collected and resuspended in an appropriate buffer for administration (e.g., normal saline).

Age and sex matched laboratory rats of a single type are divided into four groups, sham myocardial infarction (MI), adherent MSC treated, non-adherent MSC treated, and normal saline. In all but the sham MI group, ligation of the left coronary artery is performed using well known methods (see, e.g., Gnecchi et al). Briefly, animals are anesthetized and a left thoracotamy is performed under artificial respiration. The heart is accessed through the intercostal space, the pericardial sac is cut, and the heart is exteriorized through the space. The left coronary artery is legated with a silk suture about midway between the left atrium and the apex of the heart and EKG is recorded to confirm the presence of infarction. In sham operated animals, the artery is not legated. One hour after infarction, an equal number of adherent or non-adherent MSCs are injected into a total of five sites per infarct area. Normal saline is injected into the infarct area in the control animals.

Cardiac function is analyzed at regular intervals after the surgery and administration of the cells, for example by EKG. Either throughout the course of the experiment, or at the end of the experiment, rats are euthanized and hearts are excised. Analysis is performed to determine any of a number of outcomes including, but not limited to, infarct area, engraftment of MSCs into the infarct area, angiogenesis in the infarct area, and/or mRNA or protein expression. Methods for performing such analyses are known to those skilled in the art. The therapeutic effect of the cells grown in adherent culture and non-adherent culture are compared to each other and to control animals.

It is understood that comparable experiments can be performed using different animals including, for example, pigs.

The invention may be embodied in other specific forms without departing from the spirit or essential characteristics thereof The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

All references, patents, patent applications, and GenBank numbers cited are incorporated herein by reference in their entirety.

1. A method for propagation of a non-adherent culture of mesenchymal stem cells (MSCS) comprising expanding MSCs in or on a non-adherent matrix.

2. The method of claim 1, comprising encapsulation of MSCs in Matrigel™ or Hydrogel.

3. The method of claim 1, comprising the cells propagated on agarose or on Teflon®.

4. The methods of claim 1, wherein the cells are propagated in the non-adherent culture without the use of trypsin.

5. The methods of claim 1, comprising mechanical manipulation of the MSCs.

6. The method of claim 1, further comprising a biological sample containing MSCs.

7. The method of claim 6, further comprising isolating the MSCs from the biological sample containing the MSCs.

8. The method of claim 7, wherein the isolated MSCs are substantially purified.

9. The method of claim 1, wherein the MSCs are expanded at least 2-fold, 10-fold, 100-fold, 1000-fold, 10,000-fold, or 100,000 fold.

10. The method of claim 1, wherein the MSCs are suitable for administration to a subject.

11. The method of claim 10, wherein the subject is a human subject.

12. The method of claim 1, wherein the MSCs are propagated in non-adherent culture for at least a week, at least 2 weeks, at least a month, or at least 2 months.

13. A method for treatment of a subject having a disease or condition susceptible to treatment with MSCs comprising administration of MSCs grown in a non-adherent culture of claim 1.

14. The method of claim 13, wherein the disease or condition susceptible to treatment with MSCs is selected from the group consisting of muscle disease, neural disease, and vascular disease.

15. The method of claim 13, wherein the MSCs are allogenic or autologous to the subject.

16. The method of claim 13, wherein the subject is human.

17-18. (canceled)

19. A kit comprising an MSC of claim 1, and appropriate packing material.

20. The kit of claim 19, further comprising reagents or supplies for propagation of MSCs under adherent or non-adherent conditions or both.
